(19) United States
(12) Patent Application Publication
(10) Pub. No.: US 2025/0204564 A1
(43) Pub. Date: Jun. 26, 2025

(54) TASTE MODIFYING COMPOSITIONS AND USES THEREOF

(71) Applicant: Firmenich Incorporated, Plainsboro, NJ (US)

(72) Inventors: Joseph R. FOTSING, San Diego, CA (US); Guy SERVANT, San Diego, CA (US); Tanya DITSCHUN, San Diego, CA (US)

(73) Assignee: Firmenich Incorporated, Plainsboro, NJ (US)

(21) Appl. No.: 18/849,899

(22) PCT Filed: Mar. 27, 2023
(86) PCT No.: PCT/US2023/016372

§ 371 Date: Sep. 23, 2024

Related U.S. Application Data
(60) Provisional application No. 63/328,041, filed on Apr. 6, 2022.

(51) Int. Cl. A23L 27/00; A23L 27/20

(52) U.S. Cl. CPC A23L 27/86; A23L 27/2056; A23L 27/84

(57) ABSTRACT

The present disclosure generally relates to various formulations and uses of N-(7-acetyl-2,3-dihydrobenzo[b][1,4]dioxin-6-yl)-2-(4-acetylpiperazin-1-yl) acetamide (TM1), or any comestibly acceptable salts thereof. In some aspects, the disclosure provides compositions that include TM1, or a comestibly acceptable salt thereof. In some embodiments, the compositions are ingestible compositions, including, but not limited to, packaged food and beverage products. In some other aspects, the disclosure provides uses of TM1, or a comestibly acceptable salt thereof, and related methods for reducing a bitter taste of an ingestible composition, such as an ingestible composition for use in a food, beverage, oral care, nutraceutical, or pharmaceutical product.

### TECHNICAL FIELD

The present disclosure generally relates to various formulations and uses of the compound: N-(7-acetyl-2,3-dihydrobenzo[b][1,4]dioxin-6-yl)-2-(4-acetylpiperazin-1-yl)acetamide, which is also referred to herein as “TM1” or any comestibly acceptable salts thereof. In some aspects, the disclosure provides compositions that include TM1, or a comestibly acceptable salt thereof. In some embodiments, the compositions are ingestible compositions, including, but not limited to, packaged food and beverage products. In some other aspects, the disclosure provides uses of TM1, or a comestibly acceptable salt thereof, and related methods for reducing a bitter taste of an ingestible composition, such as an ingestible composition for use in a food, beverage, oral care, nutraceutical, or pharmaceutical product.

### DESCRIPTION OF RELATED ART

The taste system provides sensory information about the chemical composition of the external world. Taste transduction is one of the most sophisticated forms of chemical-triggered sensation in animals. Signaling of taste is found throughout the animal kingdom, from simple metazoans to the most complex of vertebrates. Mammals are believed to have five basic taste modalities: sweet, bitter, sour, salty, and umami.

Obesity, diabetes, and cardiovascular disease are major health concerns throughout the world, and are growing at an alarming rate. Sugar and calories are key components that can be limited to render a positive nutritional effect on health. Even so, because a number of foods contain components that agonize bitter taste receptors, consumers often use sugar and other sweeteners to offset the perception of bitter taste in such foods. Further, certain nutritionally useful foods, such as vegetable proteins, agonize bitter taste receptors. Thus, despite their nutritional value, consumers may avoid such foods because of their perceived poor taste. Further, many medicinal compounds, such as antibiotics, have a distinctly bitter taste. Therefore, nutraceutical or pharmaceutical manufacturers must employ complicated formulation technologies to permit subjects to take such medicines orally without experiencing displeasure.

But the physiological basis concerning the perception of bitter taste is still not well understood. Many bitter compounds produce their bitter taste, at least in part, by modulating certain cell surface receptors, some of which belong to a family of seven transmembrane domain receptors that interact with intracellular G proteins. These include a family of G-coupled protein receptors (GCPRs), termed T2Rs, which are found in humans and rodents. Humans express at least several dozen different receptors that fall within the T2R family. Some compounds are known to antagonize hT2 receptors. Even so, merely antagonizing the receptor is not always enough to convert otherwise bitter-tasting products into products that consumers view as tasting good. Thus, there is a continuing need to discover new formulations and new uses of bitter blocking compounds that can be used beneficially in food, beverage, oral care, nutraceutical, and pharmaceutical products.

### SUMMARY

The present disclosure generally relates to various formulations and uses of N-(7-acetyl-2,3-dihydrobenzo-[b][1,4]dioxin-6-yl)-2-(4-acetylpiperazin-1-yl) acetamide, which is also referred to herein as “TM1” or any comestibly acceptable salts thereof. In some aspects, the disclosure provides compositions that include TM1, or a comestibly acceptable salt thereof. In some embodiments, the compositions are ingestible compositions, including, but not limited to, compositions for use in food, beverage, oral care, nutraceutical, or pharmaceutical products. In some other aspects, the disclosure provides uses of TM1, or a comestibly acceptable salt thereof, and related methods for reducing a bitter taste of an ingestible composition, such as an ingestible composition for use in a food, beverage, oral care, or pharmaceutical products. N-(7-Acetyl-2,3-dihydrobenzo-[b][1,4]dioxin-6-yl)-2-(4-acetylpiperazin-1-yl)acetamide, which is also referred to herein as “TM1” and which is represented by the chemical structure

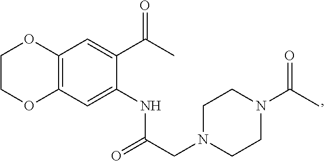 [SMILES: CC(=O)c1cc2c(cc1NC(=O)CN1CCN(C(C)=O)CC1)OCCO2]

and comestibly acceptable salts thereof, impart certain beneficial taste properties when used in combination with other sweeteners and flavor-modifying compounds, and when formulated in particular solid-state forms.

In a first aspect, the disclosure provides N-(7-acetyl-2,3-dihydrobenzo-[b][1,4]dioxin-6-yl)-2-(4-acetylpiperazin-1-yl)acetamide, or a comestibly acceptable salt thereof.

In a second aspect, the disclosure provides solid-state forms, such as crystalline polymorphs, co-crystals, and solvates of the compounds of the first aspect.

In a third aspect, the disclosure provides an ingestible composition comprising a compound of the of the first aspect or a solid-state form of the second aspect, for example, for use in various comestible products, such as food, beverage, oral care, nutraceutical, or pharmaceutical products, and in combination with one or more sweeteners, umami tastants, flavor modifiers, mouthfeel enhancers, and the like.

In a fourth aspect, the disclosure provides uses of the compounds of the first aspect, the solid-state forms of the second aspect, or the ingestible compositions of the third aspect to modify the flavor or to impart flavor to a comestible product (for example, to reduce a bitter taste of a comestible product), such as a food, beverage, oral care, nutraceutical, or pharmaceutical product. In a related aspect, the disclosure provides methods of modifying a flavor (for example, reducing a bitter taste) of a comestible product, the method comprising introducing the compounds of the first aspect, the solid-state forms of the second aspect, or the ingestible compositions of the third aspect to the comestible product. In some embodiments of these aspects, the comestible product comprises one or more bitter tastants, such as caffeine, tannins, compounds from coffee, compounds from ginseng, non-animal proteins, such as pea or soy or potato protein, animal proteins, such as whey protein or casein, citrus fruit or juices, and high-intensity sweeteners, such as saccharin, steviol glycosides, aspartame, and the like.

Further aspects, and embodiments thereof, are set forth below in the Detailed Description, the Drawings, the Abstract, and the Claims.

### BRIEF DESCRIPTION OF THE DRAWINGS

The drawings are provided for illustrative purposes only, and are not intended to describe any preferred compositions or preferred methods, or to serve as a source of any limitations on the scope of the claimed inventions.

FIG. 1 shows a chemical formula that represents the compound N-(7-acetyl-2,3-dihydrobenzo-[b][1,4]dioxin-6-yl)-2-(4-acetylpiperazin-1-yl)acetamide.

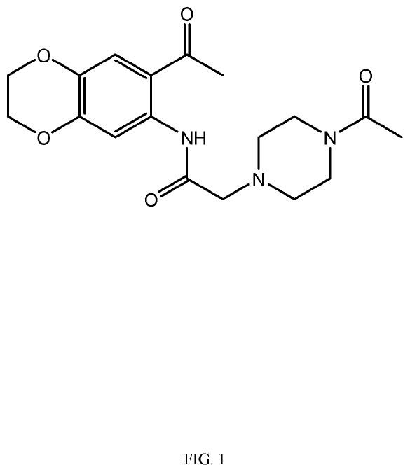

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Definitions

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary

As used herein, “solvate” means a compound formed by the interaction of one or more solvent molecules and one or more compounds described herein. In some embodiments, the solvates are ingestibly acceptable solvates, such as hydrates.

The terms a “sweetener” or a “sweet flavoring agent” or a “sweet flavor entity” or a “sweet compound” or a “sweet tastant” herein refers to a compound that elicits a detectable sweet flavor in a subject, e.g., a compound that activates a T1R2/T1R3 taste receptor in vitro.

The terms a “bitter compound” or a “bitter tastant” herein refers to a compound that elicits a detectable bitter flavor in a subject, e.g., a compound that activates one or more T2R taste receptor in vitro.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of”' refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

Chemical structures are often shown using the “skeletal” format, such that carbon atoms are not explicitly shown, and hydrogen atoms attached to carbon atoms are omitted entirely. For example, the structure

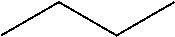

represents butane (i.e., n-butane). Furthermore, aromatic groups, such as benzene, are represented by showing one of the contributing resonance structures. For example, the structure

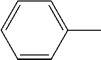

represents toluene.

Other terms are defined in other portions of this description, even though not included in this subsection.

### Flavor-Modifying Compounds

In a first aspect, the disclosure provides flavor-modifying compounds, which are compounds of the following formula:

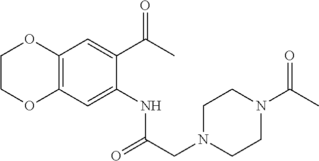 [SMILES: CC(=O)c1cc2c(cc1NC(=O)CN1CCN(C(C)=O)CC1)OCCO2]

or comestibly acceptable salts thereof. The compound of the above formula is also described herein as N-(7-acetyl-2,3-dihydrobenzo[b][1,4]dioxin-6-yl)-2-(4-acetylpiperazin-1-yl)acetamide, or by the abbreviation “TM1”.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

In some embodiments, the compounds disclosed herein are capable of forming acid and/or base salts by virtue of the presence of amino and/or carboxyl groups or groups similar thereto. Comestibly acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Physiologically acceptable salts can be formed using inorganic and organic bases. Inorganic bases from which salts can be derived include, for example, bases that contain sodium, potassium, lithium, ammonium, calcium, magnesium, iron, zinc, copper, manganese, aluminum, and the like; particularly preferred are the ammonium, potassium, sodium, calcium and magnesium salts. In some embodiments, treatment of the compounds disclosed herein with an inorganic base results in loss of a labile hydrogen from the compound to afford the salt form including an inorganic cation such as Li⁺, Na⁺, K⁺, Mg²⁺ and Ca²⁺ and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

In some embodiments, the compound is a salt of N-(7-acetyl-2,3-dihydrobenzo-[b][1,4]dioxin-6-yl)-2-(4-acetylpiperazin-1-yl)acetamide, such as a hydrochloride salt of N-(7-acetyl-2,3-dihydrobenzo-[b][1,4]dioxin-6-yl)-2-(4-acetylpiperazin-1-yl)acetamide.

### Solid State Forms and Solutions of Flavor-Modifying Compounds

In another aspect, the disclosure provides various solid-state forms of the TM1 compound or its comestibly acceptable salts.

In some embodiments, the TM1 compound or any of its comestibly acceptable salts exists as a crystalline solid, either in substantially pure form or in a formulation such as those set forth below. The crystalline solid can have any suitable polymorphic form, such as any polymorphic form obtainable via recrystallization in any suitable solvent system, according to techniques commonly used in the art of polymorph screening.

In some other embodiments, the TM1 compound or any of its comestibly acceptable salts exists as an amorphous solid or a semi-amorphous solid, meaning that it lacks any regular crystalline structure. Such solids can be generated using standard techniques, such as spray drying, and the like.

In some embodiments, the TM1 compound or any of its comestibly acceptable salts exists as a solvate, which is a pseudomorphic form of the compound in which one or more solvent molecules (such as water molecules) are taken up into the crystalline structure. Any suitable solvent or combination of solvents can be used, including, but not limited to, water, methanol, ethanol, n-propanol, isopropanol, n-butanol, 2-butanol, isobutanol, ethyl acetate, ethylene glycol, 1,2-propylene glycol, 1,3-propylene glycol, and the like. In some embodiments, the disclosure provides hydrates of the TM1 compound or its comestibly acceptable salts. Such solvates can be generated by any suitable means, such as those techniques typically used by skilled artisans in the field of polymorph and solvate screening.

In some other embodiments, the TM1 compound or any of its comestibly acceptable salts exist as a co-crystal with one or more other compounds, such as one or more other sweetener compounds. The TM1 compound or any of its comestibly acceptable salts can form a co-crystal with any suitable compound. Non-limiting examples of such suitable compounds include fructose, glucose, galactose, sucrose, lactose, maltose, allulose, sugar alcohols (such as erythritol, sorbitol, xylitol, and the like), sucralose, steviol glycosides (such as rebaudioside A, rebaudioside D, rebaudioside E, rebaudioside M, and the like natural stevioside compounds), mogrosides (such as mogroside V, isomogroside V, siamenoside I, isomogroside IVE, or the 1,6-alpha isomer of siamenoside I) and other like natural mogroside compounds), aspartame, saccharin, acesulfame K, cyclamate, inulin, isomalt, and maltitol. Such co-crystals can be generated by any suitable means, such as those set forth in U.S. Patent Application Publication No. 2018/0363074, which is incorporated herein by reference.

In some embodiments, the TM1 compound or its comestibly acceptable salts is in the form of a dry particle. Such dry particles can be formed by standard techniques in the art, such as dry granulation, wet granulation, and the like. Such particles can also contain a number of excipients, including, inert diluents, such as calcium carbonate, sodium carbonate, lactose, calcium phosphate, and sodium phosphate; granulating and disintegrating agents, such as starch, cellulosic materials, and alginic acid; binding agents, such as gelatin, guar gum, and acacia; and lubricating agents, such as magnesium stearate, stearic acid, and talc. Other excipients typically used in food and beverage products can also be included, such as typical foodstuff materials.

In some embodiments, the TM1 compound or its comestibly acceptable salts are in the form of a liquid solution or a liquid suspension. Such compositions can also include: carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. Such compositions can also include one or more coloring agents, one or more flavoring agents, and the like. Such liquid suspensions and solutions have a liquid carrier. In general, the liquid carrier comprises water. In some such cases, the liquid composition is an emulsion, such as an oil-in-water or a water-in-oil emulsion. Further, in some cases, water may be too polar to dissolve the TM1 compound to the desired concentration. In such instances, it can be desirable to introduce water-miscible solvents, such as alcohols, glycols, polyols, and the like, to the solvent to enhance solubilization of the TM1 compound.

In some embodiments, the TM1 compound, or its comestibly acceptable salts, is in the form of a solution, i.e., are solvated within a liquid carrier. In some embodiments, the liquid carrier is an aqueous carrier. In some such embodiments, the solutions comprise a comestibly acceptable salt of TM1, such as a hydrochloride sale, a potassium salt, or a sodium salt. Such solutions can be diluted to any suitable concentration.

### Formulations, Uses, and Methods

In certain aspects, the disclosure provides an ingestible composition comprising TM1 or a comestibly acceptable salt thereof. In some embodiments, the ingestible composition comprises one or more bitter tastants.

In certain aspects, the disclosure provides uses of TM1, or a comestibly acceptable salt thereof, to reduce a bitter taste of an ingestible composition. In certain related aspects, the disclosure provides methods of reducing a bitter taste of an ingestible composition, the methods comprising introducing TM1, or a comestibly acceptable salt thereof, to the ingestible composition. In some embodiments, the ingestible composition comprises a bitter tastant.

In certain aspects, the disclosure provides uses of TM1, or a comestibly acceptable salt thereof, to reduce an astringent taste of an ingestible composition. In certain related aspects, the disclosure provides methods of reducing an astringent taste of an ingestible composition, the methods comprising introducing TM1, or a comestibly acceptable salt thereof, to the ingestible composition. In some embodiments, the ingestible composition comprises an astringent tastant.

In certain aspects, the disclosure provides uses of TM1, or a comestibly acceptable salt thereof, to enhance a mouthfeel of an ingestible composition. In certain related aspects, the disclosure provides methods of enhancing a mouthfeel of an ingestible composition, the methods comprising introducing TM1, or a comestibly acceptable salt thereof, to the ingestible composition.

In certain aspects, the disclosure provides uses of TM1, or a comestibly acceptable salt thereof, to enhance a sweet taste of an ingestible composition. In certain related aspects, the disclosure provides methods of enhancing a sweet taste of an ingestible composition, the methods comprising introducing TM1, or a comestibly acceptable salt thereof, to the ingestible composition. In some embodiments, the ingestible composition comprises a sweetener, such as a high-intensity sweetener.

In certain aspects, the disclosure provides uses of TM1, or a comestibly acceptable salt thereof, to reduce sugar content of an ingestible composition. In certain related aspects, the disclosure provides methods of reducing sugar content of an ingestible composition, the methods comprising introducing TM1, or a comestibly acceptable salt thereof, to the ingestible composition. In some embodiments, the sugar is sucrose, fructose (for example, high-fructose corn syrup), glucose, or a combination thereof.

The ingestible compositions referenced above can include TM1, or a comestibly acceptable salt thereof, in any suitable concentration. In certain embodiments, the ingestible composition comprises bitter compounds and the TM1 compound (or comestibly acceptable salts thereof). In some embodiments, the concentration of the TM1 compound, or a comestibly acceptable salt thereof, in the ingestible composition ranges from 0.1 ppm to 1000 ppm, or from 0.1 ppm to 900 ppm, or from 0.1 ppm to 800 ppm, or from 0.1 ppm to 700 ppm, or from 0.1 ppm to 600 ppm, or from 0.1 ppm to 500 ppm, or from 0.1 ppm to 400 ppm, or from 0.1 ppm to 300 ppm, or from 0.1 ppm to 200 ppm, or from 0.1 ppm to 100 ppm, or from 0.1 ppm to 50 ppm, or from 0.1 ppm to 25 ppm, or from 0.1 ppm to 10 ppm, or from 1 ppm to 1000 ppm, or from 1 ppm to 900 ppm, or from 1 ppm to 800 ppm, or from 1 ppm to 700 ppm, or from 1 ppm to 600 ppm, or from 1 ppm to 500 ppm, or from 1 ppm to 400 ppm, or from 1 ppm to 300 ppm, or from 1 ppm to 200 ppm, or from 1 ppm to 100 ppm, or from 1 ppm to 50 ppm, or from 1 ppm to 25 ppm, or from 1 ppm to 10 ppm. Such ingestible compositions can be in any suitable form. In some embodiments, the ingestible composition is a food product, such as any of those specifically listed below. In other embodiments, the ingestible composition is a beverage product, such as a soda, and the like. In other embodiments, the ingestible composition is an oral care product, such as toothpaste, mouthwash, whitening composition, and the like. In other embodiments, the ingestible composition is a nutraceutical product. In other embodiments, the ingestible composition is a pharmaceutical product, such as an OTC or prescription drug product. The bitter compounds include, but are not limited to, active pharmaceutical ingredients (APIs), tannins (such as those in coffee, tea, or wine), ginseng, vitamins, minerals, limonin or nomelin (such as found in citrus juices), caffeine, quinine, catechins, polyphenols, potassium chloride, menthol, or plant starches or proteins (such as pea protein, soy protein, or potato protein), algal proteins or starches, fungal proteins or starches, or alcohol.

In certain particular embodiments, the ingestible composition comprises animal products (such as animal proteins, which can be replaced by starches or proteins derived from plants, algae, or fungi), and the TM1 compound (or comestibly acceptable salts thereof). In some such embodiments, the introduction of the TM1 compound (or comestibly acceptable salt thereof) permits one to use less animal product (such as more than 10% less, more than 20% less, more than 30% less, more than 40% less, more than 50% less, more than 60% less, or more than 70% less, or more than 80% less, or more than 90% less) and still achieve a taste characteristic of a comparable product that employs a higher concentration of animal products. In some related embodiments, the use of the TM1 compound, or its comestibly acceptable salts, permits the elimination of animal products from the composition. Such ingestible compositions can be in any suitable form. In some embodiments, the ingestible composition is a food product, such as any of those specifically listed below. In other embodiments, the ingestible composition is a beverage product, such as a soda, and the like. The animal products can be any suitable animal product, such as cheese, milk, meat broth (such as beef broth, pork broth, chicken broth, turkey broth, duck broth, lamb broth, goat broth, rabbit broth, and the like), eggs, bone broth, bone marrow, meat (such as beef, pork, chicken, lamb, goat, turkey, duck, rabbit, and the like), butter, and animal skin.

In certain particular embodiments, the ingestible composition comprises sodium (i.e., sodium cation, which can be replaced by potassium cation), and the TM1 compound (or comestibly acceptable salts thereof). In some such embodiments, the introduction of the TM1 compound (or comestibly acceptable salt thereof) permits one to use less sodium (such as more than 10% less, more than 20% less, more than 30% less, more than 40% less, more than 50% less, more than 60% less, or more than 70% less, or more than 80% less, or more than 90% less) and still achieve a taste characteristic of a comparable product that employs a higher concentration of sodium. In some related embodiments, the use of the TM1 compound, or its comestibly acceptable salts, permits the elimination of sodium from the composition. Such ingestible compositions can be in any suitable form. In some embodiments, the ingestible composition is a food product, such as any of those specifically listed below. In other embodiments, the ingestible composition is a beverage product, such as a soda, and the like. The sodium can be any suitable sodium source, such as table salt, sea salt, soy sauce, fish sauce, shrimp paste, butter, miso, and Worcestershire sauce.

In some instances, one may be able to reduce the amount of sugar in a product by reducing bitter taste. In some such embodiments, the introduction of the TM1 compound (or salt) permits one to use less sugar (such as more than 10% less, more than 20% less, more than 30% less, more than 40% less, more than 50% less, more than 60% less, or more than 70% less) and still achieve a taste characteristic of a comparable product that employs more sugar. The sugar can be any suitable sugar or combination of sugars, including, without limitation, sucrose, fructose (for example, high-fructose corn syrup), glucose, allulose, or any combinations thereof. In some embodiments, the ingestible composition is a food product, such as any of those specifically listed below. In other embodiments, the ingestible composition is a beverage product, such as a soda, and the like.

In some instances, one may be able to reduce the amount of a non-sugar sweetener in a product by reducing bitter taste. In some such embodiments, the introduction of the TM1 compound (or salt) permits one to use less non-sugar sweetener (such as more than 10% less, more than 20% less, more than 30% less, more than 40% less, more than 50% less, more than 60% less, or more than 70% less) and still achieve a taste characteristic of a comparable product that employs more non-sugar sweetener. The non-sugar sweetener can be any suitable sugar or combination of sugars, including, without limitation, sucralose, rebaudiosides (such as rebaudioside A, rebaudioside D, rebaudioside E, rebaudioside M, or any combination thereof), acesulfame potassium, sugar alcohols (such as erythritol), aspartame, neotame, cyclamate, mogrosides (such as mogroside III, mogroside IV, mogroside V, siamenoside I, isomogroside V, mogroside IVE, isomogroside IV, isomogroside IVE, mogroside IIIE, 11-oxomogroside V, the 1,6-alpha isomer of siamenoside I, or any combinations thereof), or any combinations thereof. In some embodiments, the ingestible composition is a food product, such as any of those specifically listed below. In other embodiments, the ingestible composition is a beverage product, such as a soda, and the like.

In certain embodiments of any aspects and embodiments set forth herein that refer to an ingestible composition, the ingestible composition is a non-naturally-occurring product, such as a composition specifically manufactured for the production of a flavored product, such as food or beverage product.

In general, compounds as disclosed and described herein, individually or in combination, can be provided in a composition, such as an ingestible composition. In one embodiment, compounds as disclosed and described herein, individually or in combination, can impart a more sugar-like temporal profile or flavor profile to a sweetener composition by combining one or more of the compounds as disclosed and described herein with one or more sweeteners in the sweetener composition. In another embodiment, compounds as disclosed and described herein, individually or in combination, can increase or enhance the sweet taste of a composition by contacting the composition thereof with the compounds as disclosed and described herein to form a modified composition.

Thus, in some embodiments, the compositions set forth in any of the foregoing aspects (including in any uses or methods), comprise TM1, or a comestibly acceptable salt thereof, and a sweetener. In some embodiments, the composition further comprises a vehicle. In some embodiments, the vehicle is water

The sweetener can be present in the ingestible composition in any suitable concentration. For example, in some embodiments, the sweetener is present in an amount from about 0.1% to about 12% by weight. In some embodiments, the sweetener is present in an amount from about 0.2% to about 10% by weight. In some embodiments, the sweetener is present in an amount from about 0.3% to about 8% by weight. In some embodiments, the sweetener is present in an amount from about 0.4% to about 6% by weight. In some embodiments, the sweetener is present in an amount from about 0.5% to about 5% by weight. In some embodiments, the sweetener is present in an amount from about 1% to about 2% by weight. In some embodiments, the sweetener is present in an amount from about 0.1% to about 5% by weight. In some embodiments, the sweetener is present in an amount from about 0.1% to about 4% by weight. In some embodiments, the sweetener is present in an amount from about 0.1% to about 3% by weight. In some embodiments, the sweetener is present in an amount from about 0.1% to about 2% by weight. In some embodiments, the sweetener is present in an amount from about 0.1% to about 1% by weight. In some embodiments, the sweetener is present in an amount from about 0.1% to about 0.5% by weight. In some embodiments, the sweetener is present in an amount from about 0.5% to about 10% by weight. In some embodiments, the sweetener is present in an amount from about 2% to about 8% by weight. In some further embodiments of the embodiments set forth in this paragraph, the sweetener is sucrose, fructose, glucose, xylitol, erythritol, or combinations thereof.

In some other embodiments, lower concentrations of sweetener may be more appropriate. For example, in some embodiments, the sweetener is present in an amount from 10 ppm to 1000 ppm. In some embodiments, the sweetener is present in an amount from 20 ppm to 800 ppm. In some embodiments, the sweetener is present in an amount from 30 ppm to 600 ppm. In some embodiments, the sweetener is present in an amount from 40 ppm to 500 ppm. In some embodiments, the sweetener is present in an amount from 50 ppm to 400 ppm. In some embodiments, the sweetener is present in an amount from 50 ppm to 300 ppm. In some embodiments, the sweetener is present in an amount from 50 ppm to 200 ppm. In some embodiments, the sweetener is present in an amount from 50 ppm to 150 ppm. In some further embodiments of the embodiments set forth in this paragraph, the sweetener is a steviol glycoside, a mogroside, a derivative of either of the foregoing, such as glycoside derivatives (e.g., glucosylates), or any combination thereof.

The compositions can include any suitable sweeteners or combination of sweeteners. In some embodiments, the sweetener is a common saccharide sweeteners, such as sucrose, fructose, glucose, and sweetener compositions comprising natural sugars, such as corn syrup (including high fructose corn syrup) or other syrups or sweetener concentrates derived from natural fruit and vegetable sources. In some embodiments, the sweetener is sucrose, fructose, or a combination thereof. In some embodiments, the sweetener is sucrose. In some other embodiments, the sweetener is selected from rare natural sugars including D-allose, D-psicose, L-ribose, D-tagatose, L-glucose, L-fucose, L-arbinose, D-turanose, and D-leucrose. In some embodiments, the sweetener is selected from semi-synthetic “sugar alcohol” sweeteners such as erythritol, isomalt, lactitol, mannitol, sorbitol, xylitol, maltodextrin, and the like. In some embodiments, the sweetener is selected from artificial sweeteners such as aspartame, saccharin, acesulfame-K, cyclamate, sucralose, and alitame. In some embodiments, the sweetener is selected from the group consisting of cyclamic acid, mogroside, tagatose, maltose, galactose, mannose, sucrose, fructose, lactose, allulose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A, other sweet Stevia-based glycosides, chemically modified steviol glycosides (such as glucosylated steviol glycosides), mogrosides, chemically modified mogrosides (such as glucosylated mogrosides), carrelame and other guanidine-based sweeteners. In some embodiments, the sweetener is a combination of two or more of the sweeteners set forth in this paragraph. In some embodiments, the sweetener may combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a sugar. In some embodiments, the sugar is cane sugar. In some embodiments, the sugar is beet sugar. In some embodiments, the sugar may be sucrose, fructose, glucose or combinations thereof. In some embodiments, the sugar may be sucrose. In some embodiments, the sugar may be a combination of fructose and glucose.

The sweetener can also include, for example, sweetener compositions comprising one or more natural or synthetic carbohydrate, such as corn syrup, high fructose corn syrup, high maltose corn syrup, glucose syrup, sucralose syrup, hydrogenated glucose syrup (HGS), hydrogenated starch hydrolyzate (HSH), or other syrups or sweetener concentrates derived from natural fruit and vegetable sources, or semi-synthetic “sugar alcohol” sweeteners such as polyols. Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, isomaltulose, maltodextrin, and the like, and sugar alcohols or any other carbohydrates or combinations thereof capable of being reduced which do not adversely affect taste.

The sweetener may be a natural or synthetic sweetener that includes, but is not limited to, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

In still other embodiments, the sweetener can be a chemically or enzymatically modified natural high potency sweetener. Modified natural high potency sweeteners include glycosylated natural high potency sweetener such as glucosyl-, galactosyl-, or fructosyl-derivatives containing 1-50 glycosidic residues. Glycosylated natural high potency sweeteners may be prepared by enzymatic transglycosylation reaction catalyzed by various enzymes possessing transglycosylating activity. In some embodiments, the modified sweetener can be substituted or unsubstituted.

Additional sweeteners also include combinations of any two or more of any of the aforementioned sweeteners. In some embodiments, the sweetener may comprise combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a caloric sweetener, such as sucrose, fructose, xylitol, erythritol, or combinations thereof. In some embodiments, the ingestible compositions are free (or, in some embodiments) substantially free of stevia-derived sweeteners, such as steviol glycosides, glucosylated steviol glycosides, or rebaudiosides. For example, in some embodiments, the ingestible compositions are either free of stevia-derived sweeteners or comprise stevia-derived sweeteners in a concentration of no more than 1000 ppm, or no more than 500 ppm, or no more than 200 ppm, or no more than 100 ppm, or no more than 50 ppm, or no more than 20 ppm, or no more than 10 ppm, or no more than 5 ppm, or no more than 3 ppm, or no more than 1 ppm.

The ingestible compositions can, in certain embodiments, comprise any additional ingredients or combination of ingredients as are commonly used in food and beverage products, including, but not limited to:
acids, including, for example citric acid, phosphoric acid, ascorbic acid, sodium acid sulfate, lactic acid, or tartaric acid;
bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi);
coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;
preservatives, including, for example sodium benzoate, potassium benzoate, potassium sorbate, sodium metabisulfate, sorbic acid, or benzoic acid;
antioxidants including, for example ascorbic acid, calcium disodium EDTA, alpha tocopherols, mixed tocopherols, rosemary extract, grape seed extract, resveratrol, or sodium hexametaphosphate;
vitamins or functional ingredients including, for example resveratrol, Co-Q10, omega 3 fatty acids, theanine, choline chloride (citocoline), fibersol, inulin (chicory root), taurine, panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;
clouding agents, including, for example ester gun, brominated vegetable oil (BVO), or sucrose acetate isobutyrate (SAIB);
buffers, including, for example sodium citrate, potassium citrate, or salt;
flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate; or
starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan.

The ingestible compositions or sweetener concentrates can have any suitable pH. In some embodiments, the flavor-modifying compounds enhance the sweetness of a sweetener under a broad range of pH, e.g., from lower pH to neutral pH. The lower and neutral pH includes, but is not limited to, a pH from 1.5 to 9.0, or from 2.5 to 8.5; from 3.0 to 8.0; from 3.5 to 7.5; and from 4.0 to 7. In certain embodiments, compounds as disclosed and described herein, individually or in combination, can enhance the perceived sweetness of a fixed concentration of a sweetener in taste tests at a compound concentration of 50 μM, 40 μM, 30 μM, 20 μM, or 10 μM at both low to neutral pH value. In certain embodiments, the enhancement factor of the compounds as disclosed and described herein, individually or in combination, at the lower pH is substantially similar to the enhancement factor of the compounds at neutral pH. Such consistent sweet enhancing property under a broad range of pH allow a broad use in a wide variety of foods and beverages of the compounds as disclosed and described herein, individually or in combination.

In some embodiments, ingestible compositions disclosed herein comprise the TM1 compound, or any comestibly acceptable salts thereof, combined with one or more umami or kokumi tastants. Such umami or kokumi tastants include, but are not limited to, N-(heptan-4-yl)benzo[d][1,3]dioxole-5-carboxamide, N¹-(2,4-dimethoxybenzyl)-N²-(2-(pyridin-2-yl)ethyl)oxalamide, alkyl amides, glutamates (such as monosodium glutamate (MSG)), arginates, purinic ribotides (such as inosine monophosphate (IMP), adenosine monophosphate (AMP), guanosine monophosphate (GMP), and sodium salts thereof), amino acids (such as L-threanine), oligopeptides (such as glutamyl oligopeptides), cheeses and cheese extracts, yeast extracts, and alcohol. The TM1 compound (or comestibly acceptable salts thereof) may be used in combination with such umami or kokumi tastants in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some embodiments, ingestible compositions disclosed herein comprise the TM1 compound, or any comestibly acceptable salts thereof, combined with one or more bitter tastants. Such bitter tastants include, but are not limited to, active pharmaceutical ingredients (APIs), tannins (such as those in coffee, tea, or wine), ginseng, vitamins, minerals, limonin or nomelin (such as found in citrus juices), caffeine, quinine, catechins, polyphenols, potassium chloride, menthol, other commonly used oral care ingredients, cooling agents (such as N-ethyl-N-(thiophen-2-ylmethyl)-2-(p-tolyloxy)acetamide, N-(1H-pyrazol-3-yl)-N-(thiophen-2-ylmethyl)-2-(p-tolyloxy)acetamide, 2-(4-fluorophenoxy)-N-(1H-pyrazol-3-yl)-N-(thiophen-2-ylmethyl)acetamide, 2-(2-hydroxy-4-methylphenoxy)-N-(1H-pyrazol-3-yl)-N-(thiophen-2-ylmethyl)-acetamide, 2-((2,3-dihydro-1H-inden-5-yl)oxy)-N-(1H-pyrazol-3-yl)-N-(thiophen-2-ylmethyl)-acetamide, 2-((2,3-dihydro-1H-inden-5-yl)oxy)-N-(1H-pyrazol-3-yl)-N-(thiazol-5-ylmethyl)-acetamide, and 2-((5-methoxybenzofuran-2-yl)oxy)-N-(1H-pyrazol-3-yl)-N-(thiophen-2-ylmethyl)-acetamide), plant starches or proteins (such as pea protein, soy protein, chickpea protein, or potato protein), algal proteins or starches, fungal proteins or starches, or alcohol, high-intensity sweeteners, such as saccharin, steviol glycosides, mogrosides, aspartame, and the like. The TM1 compound (or comestibly acceptable salts thereof) may be used in combination with such bitter tastants in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

The ingestible compositions set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness, compounds that enhance umami, compounds that reduce sourness or licorice taste, compounds that enhance saltiness, compounds that enhance a cooling effect, or any combinations of the foregoing.

Thus, in some embodiments, ingestible compositions disclosed herein comprise the TM1 compound, or any comestibly acceptable salts thereof, and one or more sweetness enhancing compounds. Such sweetness enhancing compounds include, but are not limited to, 3-((4-amino-2,2-dioxo-1H-benzo[c][1,2,6]thiadiazin-5-yl)oxy)-2,2-dimethyl-N-propyl-propanamide, N-(1-((4-amino-2,2-dioxo-1H-benzo[c][1,2,6]thiadiazin-5-yl)oxy)-2-methyl-propan-2-yl)isonicotinamide, 4-amino-5,6-dimethylthieno[2,3-d]pyrimidin-2(1H)-one, hesperitin dihydrochalcone, hesperitin dihydrochalcone-4′-O′glucoside, neohesperitin dihydrochalcone, naringenin, naringin, phloretin, glucosylated steviol glycosides, trilobatin, eriodictyol, homoeriodictyol, brazzein, (2R,3R)-3-acetoxy-5,7,4′-trihydroxyflavanone, (2R,3R)-3-acetoxy-5,7,3′-trihydroxy-4′-methoxyflavanone, rubusosides, or compounds such as those set forth in U.S. Pat. Nos. 8,541,421; 8,815,956; 9,834,544; 8,592,592; 8,877,922; 9,000,054; and 9,000,051, as well as U.S. Patent Application Publication No. 2017/0119032. The TM1 compound (or comestibly acceptable salts thereof) may be used in combination with such other sweetness enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1. In some embodiments of any of the preceding embodiments, the TM1 compound (or any comestibly acceptable salts thereof) is combined with glucosylated steviol glycosides in any of the above ratios. As used herein, the term “glucosylated steviol glycoside” refers to the product of enzymatically glucosylating natural steviol glycoside compounds. The glucosylation generally occurs through a glycosidic bond, such as an α-1,2 bond, an α-1,4 bond, an α-1.6 bond, a β-1,2 bond, a β-1,4 bond, a β-1,6 bond, and so forth.

In some further embodiments, ingestible compositions disclosed herein comprise the TM1 compound, or any comestibly acceptable salts thereof, combined with one or more umami or kokumi enhancing compounds. Such umami enhancing compounds include, but are not limited to, naturally derived compounds, such as (E)-3-(3,4-dimethoxyphenyl)-N-(4-methoxyphenethyl)acrylamide, or synthetic compounds, such as N-(heptan-4-yl)-benzo[d][1,3]dioxole-5-carboxamide, N¹-(2,4-dimethoxybenzyl)-N²-(2-(pyridin-2-yl)ethyl)-oxalamide, alkyl amides, or any other compounds set forth in U.S. Pat. Nos. 8,735,081; 8,124,121; and 8,968,708. The TM1 compound (or comestibly acceptable salts thereof) may be used in combination with such umami enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the TM1 compound, or any comestibly acceptable salts thereof, combined with one or more cooling enhancing compounds. Such cooling enhancing compounds include, but are not limited to, naturally derived compounds, such as menthol or analogs thereof, or synthetic compounds, such as any compounds set forth in U.S. Pat. Nos. 9,394,287 and 10,421,727. The TM1 compound (or comestibly acceptable salts thereof) may be used in combination with such umami enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1. In some embodiments, the comestible composition comprises: N-ethyl-N-(thiophen-2-ylmethyl)-2-(p-tolyloxy)acetamide; N-(1H-pyrazol-3-yl)-N-(thiophen-2-ylmethyl)-2-(p-tolyloxy)acetamide; 2-(4-fluorophenoxy)-N-(1H-pyrazol-3-yl)-N-(thiophen-2-ylmethyl)acetamide; 2-(2-hydroxy-4-methylphenoxy)-N-(1H-pyrazol-3-yl)-N-(thiophen-2-ylmethyl)-acetamide; 2-((2,3-dihydro-1H-inden-5-yl)oxy)-N-(1H-pyrazol-3-yl)-N-(thiophen-2-ylmethyl)-acetamide; 2-((2,3-dihydro-1H-inden-5-yl)oxy)-N-(1H-pyrazol-3-yl)-N-(thiazol-5-ylmethyl)-acetamide; 2-((5-methoxybenzofuran-2-yl)oxy)-N-(1H-pyrazol-3-yl)-N-(thiophen-2-ylmethyl)-acetamide, or any combination thereof in any of the aforementioned ratios. In some embodiments, such comestible compositions comprise menthol or a menthol analogue. In some further embodiments, such comestible compositions are compositions for use in oral care products, such as toothpaste, mouthwash, whitening compositions, dentifrices, and the like.

In some further embodiments, ingestible compositions disclosed herein comprise the TM1 compound, or any comestibly acceptable salts thereof, combined with one or more other bitterness blocking compounds. Such bitterness blocking compounds include, but are not limited to 3-(1-((3,5-dimethylisoxazol-4-yl)methyl)-1H-pyrazol-4-yl)-1-(3-hydroxybenzyl)-imidazolidine-2,4-dione, or other compounds set forth in U.S. Pat. Nos. 8,076,491; 8,445,692; and 9,247,759, or in PCT Publication No. WO 2020/033669. The TM1 compound (or comestibly acceptable salts thereof) may be used in combination with such bitterness blockers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the TM1 compound, or any comestibly acceptable salts thereof, combined with one or more sour taste modulating compounds. The TM1 compound (or comestibly acceptable salts thereof) may be used in combination with such sour taste modulating compounds in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the TM1 compound, or any comestibly acceptable salts thereof, combined with one or more mouthfeel modifying compounds. Such mouthfeel modifying compounds include, but are not limited to, tannins, cellulosic materials, bamboo powder, and the like. The TM1 compound (or comestibly acceptable salts thereof) may be used in combination with such mouthfeel enhancers in any suitable ratio (w/w) ranging from 1:1000 to 1000:1, or from 1:100 to 100:1, or fro, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some further embodiments, ingestible compositions disclosed herein comprise the TM1 compound, or any comestibly acceptable salts thereof, combined with one or more flavor masking compounds. Such flavor masking compounds include, but are not limited to, cellulosic materials, materials extracted from fungus, materials extracted from plants, citric acid, carbonic acid (or carbonates), and the like. The TM1 compound (or comestibly acceptable salts thereof) may be used in combination with such mouthfeel enhancers in any suitable ratio (w/w) ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1.

In some aspects related to the preceding aspects and embodiments, the disclosure provides uses of the TM1 compound (or comestibly acceptable salts thereof) to enhance the flavor of a flavored composition, such as a flavored article. Such flavored compositions can use any suitable flavors, such as fruit flavors, meat flavors, vegetable flavors, and the like. In some embodiments, the flavored composition is a soup or broth, or a chip, or a beverage.

### Flavored Products and Concentrates

In certain aspects, the disclosure provides flavored products comprising any compositions of the preceding aspects. In some embodiments, the flavored products are beverage products, such as soda, flavored water, tea, and the like. In some other embodiments, the flavored products are food products, such as yogurt. In some other embodiments, the flavored product is an oral care product, such as toothpaste, mouthwash, whitening formulations, and the like. In some other embodiments, the flavored product is a nutraceutical product or a pharmaceutical product.

In embodiments where the flavored product is a beverage, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some embodiments, the beverage may be a soft drink.

In certain embodiments of any aspects and embodiments set forth herein that refer to an flavored product, the flavored product is a non-naturally-occurring product, such as a packaged food or beverage product.

Further non-limiting examples of food and beverage products or formulations include sweet coatings, frostings, or glazes for such products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionery category generally refers to edible product that is sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to jams and preserves, honey, chocolate spreads, nut based spreads, and yeast based spreads.

The Dairy Product category generally refers to edible product produced from mammal's milk. Examples of dairy product include, but are not limited to drinking milk products, cheese, yoghurt and sour milk drinks, and other dairy products.

Additional examples for flavored products, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary ingestible compositions also include breakfast cereals, sweet beverages or solid or liquid concentrate compositions for preparing beverages, ideally so as to enable the reduction in concentration of previously known saccharide sweeteners, or artificial sweeteners.

Some embodiments provide a chewable composition that may or may not be intended to be swallowed. In some embodiments, the chewable composition may be gum, chewing gum, sugarized gum, sugar-free gum, functional gum, bubble gum including compounds as disclosed and described herein, individually or in combination.

In some embodiments, the ingestible compositions set forth above may be provided in a flavoring concentrate formulation, e.g., suitable for subsequent processing to produce a ready-to-use (i.e., ready-to-serve) product. By “a flavoring concentrate formulation”, it is meant a formulation which should be reconstituted with one or more diluting medium to become a ready-to-use composition. The term “ready-to-use composition” is used herein interchangeably with “ingestible composition”, which denotes any substance that, either alone or together with another substance, can be taken by mouth whether intended for consumption or not. In one embodiment, the ready-to-use composition includes a composition that can be directly consumed by a human or animal. The flavoring concentrate formulation is typically used by mixing with or diluted by one or more diluting medium, e.g., any consumable or ingestible ingredient or product, to impart or modify one or more flavors to the diluting medium. Such a use process is often referred to as reconstitution. The reconstitution can be conducted in a household setting or an industrial setting. For example, a frozen fruit juice concentrate can be reconstituted with water or other aqueous medium by a consumer in a kitchen to obtain the ready-to-use fruit juice beverage. In another example, a soft drink syrup concentrate can be reconstituted with water or other aqueous medium by a manufacturer in large industrial scales to produce the ready-to-use soft drinks. Since the flavoring concentrate formulation has the flavoring agent or flavor modifying agent in a concentration higher than the ready-to-use composition, the flavoring concentrate formulation is typically not suitable for being consumed directly without reconstitution. There are many benefits of using and producing a flavoring concentrate formulation. For example, one benefit is the reduction in weight and volume for transportation as the flavoring concentrate formulation can be reconstituted at the time of usage by the addition of suitable solvent, solid or liquid.

In one embodiment, the flavoring concentrate formulation or ingestible composition comprises i) the TM1 compound, or a comestibly acceotable salt thereof; ii) a carrier; and iii) optionally at least one adjuvant. The term “carrier” denotes a usually inactive accessory substance, such as solvents, binders, or other inert medium, which is used in combination with the present compound and one or more optional adjuvants to form the formulation. For example, water or starch can be a carrier for a flavoring concentrate formulation. In some embodiments, the carrier is the same as the diluting medium for reconstituting the flavoring concentrate formulation; and in other embodiments, the carrier is different from the diluting medium. The term “carrier” as used herein includes, but is not limited to, ingestibly acceptable carrier.

The term “adjuvant” denotes an additive which supplements, stabilizes, maintains, or enhances the intended function or effectiveness of the active ingredient, such as the compound of the present invention. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more sweeteners. The one or more sweeteners can be any of the sweeteners described in this application. In another embodiment, the at least one adjuvant comprises one or more ingredients selected from the group consisting of a emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

In one embodiment, the present flavoring concentrate formulation can be in a form selected from the group consisting of liquid including solution and suspension, solid, foamy material, paste, gel, cream, and a combination thereof, such as a liquid containing certain amount of solid contents. In one embodiment, the flavoring concentrate formulation is in form of a liquid including aqueous-based and nonaqueous-based. In some embodiments, the present flavoring concentrate formulation can be carbonated or non-carbonated.

The flavoring concentrate formulation may further comprise a freezing point depressant, nucleating agent, or both as the at least one adjuvant. The freezing point depressant is an ingestibly acceptable compound or agent which can depress the freezing point of a liquid or solvent to which the compound or agent is added. That is, a liquid or solution containing the freezing point depressant has a lower freezing point than the liquid or solvent without the freezing point depressant. In addition to depress the onset freezing point, the freezing point depressant may also lower the water activity of the flavoring concentrate formulation. The examples of the freezing point depressant include, but are not limited to, carbohydrates, oils, ethyl alcohol, polyol, e.g., glycerol, and combinations thereof. The nucleating agent denotes an ingestibly acceptable compound or agent which is able to facilitate nucleation. The presence of nucleating agent in the flavoring concentrate formulation can improve the mouthfeel of the frozen Blushes of a frozen slush and to help maintain the physical properties and performance of the slush at freezing temperatures by increasing the number of desirable ice crystallization centers. Examples of nucleating agents include, but are not limited to, calcium silicate, calcium carbonate, titanium dioxide, and combinations thereof.

In one embodiment, the flavoring concentrate formulation is formulated to have a low water activity for extended shelf life. Water activity is the ratio of the vapor pressure of water in a formulation to the vapor pressure of pure water at the same temperature. In one embodiment, the flavoring concentrate formulation has a water activity of less than about 0.85. In another embodiment, the flavoring concentrate formulation has a water activity of less than about 0.80. In another embodiment, the flavoring concentrate formulation has a water activity of less than about 0.75.

In one embodiment, the flavoring concentrate formulation has the present compound in a concentration that is at least 2 times of the concentration of the compound in a ready-to-use composition. In one embodiment, the flavoring concentrate formulation has the present compound in a concentration that is at least 5 times of the concentration of the compound in a ready-to-use composition. In one embodiment, the flavoring concentrate formulation has the present compound in a concentration that is at least 10 times of the concentration of the compound in a ready-to-use composition. In one embodiment, the flavoring concentrate formulation has the present compound in a concentration that is at least 15 times of the concentration of the compound in a ready-to-use composition. In one embodiment, the flavoring concentrate formulation has the present compound in a concentration that is at least 20 times of the concentration of the compound in a ready-to-use composition. In one embodiment, the flavoring concentrate formulation has the present compound in a concentration that is at least 30 times of the concentration of the compound in a ready-to-use composition. In one embodiment, the flavoring concentrate formulation has the present compound in a concentration that is at least 40 times of the concentration of the compound in a ready-to-use composition. In one embodiment, the flavoring concentrate formulation has the present compound in a concentration that is at least 50 times of the concentration of the compound in a ready-to-use composition. In one embodiment, the flavoring concentrate formulation has the present compound in a concentration that is at least 60 times of the concentration of the compound in a ready-to-use composition. In one embodiment, the flavoring concentrate formulation has the present compound in a concentration that is up to 100 times of the concentration of the compound in a ready-to-use composition.

### Tabletop Compositions

In some further aspects, the disclosure provides a tabletop flavoring composition comprising: (a) at least one flavoring composition, which, in addition to comprising a flavor (according to any of the preceding aspects and embodiments thereof) also comprises the TM1 compound, or a comestibly acceptable salt thereof; and (b) at least one bulking agent.

The tabletop flavoring composition may take any suitable form including, but not limited to, an amorphous solid, a crystal, a powder, a tablet, a liquid, a cube, a glace or coating, a granulated product, an encapsulated form abound to or coated on to carriers/particles, wet or dried, or combinations thereof.

The tabletop flavoring composition may contain further additives known to those skilled in the art. These additives include but are not limited to bubble forming agents, bulking agents, carriers, fibers, sugar alcohols, oligosaccharides, sugars, high intensity sweeteners, nutritive sweeteners, flavorings, flavor enhancers, flavor stabilizers, acidulants, anti-caking and free-flow agents. Such additives are for example described by H. Mitchell (H. Mitchell, “Sweeteners and Sugar Alternatives in Food Technology”, Blackwell Publishing Ltd, 2006, which is incorporated herein by reference in its entirety). As used herein, the term “flavorings” may include those flavors known to the skilled person, such as natural and artificial flavors. These flavorings may be chosen from synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, yuzu, sudachi, and fruit essences including apple, pear, peach, grape, blueberry, strawberry, raspberry, cherry, plum, pineapple, watermelon, apricot, banana, melon, apricot, ume, cherry, raspberry, blackberry, tropical fruit, mango, mangosteen, pomegranate, papaya and so forth. Other potential flavors include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with cooling agents.

Flavors may also provide breath freshening properties, particularly the mint flavors when used in combination with cooling agents. These flavorings may be used in liquid or solid form and may be used individually or in admixture. Other useful flavorings include aldehydes and esters such as cinnamyl acetate, cinnamaldehyde, citral diethylacetal, dihydrocarvyl acetate, eugenyl formate, p-methylamisol, and so forth may be used. Generally any flavoring or food additive such as those described in Chemicals Used in Food Processing, publication 1274, pages 63-258, by the National Academy of Sciences, may be used. This publication is incorporated herein by reference.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), cherry, grape, strawberry shortcake, and mixtures thereof. These listings of flavorings are merely exemplary and are not meant to limit either the term “flavoring” or the scope of the disclosure generally.

In some embodiments, the flavoring may be employed in either liquid form and/or dried form. When employed in the latter form, suitable drying means such as spray drying the oil may be used. Alternatively, the flavoring may be absorbed onto water soluble materials, such as cellulose, starch, sugar, maltodextrin, gum arabic and so forth or may be encapsulated. The actual techniques for preparing such dried forms are well-known.

In some embodiments, the tabletop sweetener can be made to be similar to brown sugar. In such embodiments, compounds imparting brown notes can be added to the composition to make it taste more similar to brown sugar.

In some embodiments, the flavorings may be used in many distinct physical forms well-known in the art to provide an initial burst of flavor and/or a prolonged sensation of flavor. Without being limited thereto, such physical forms include free forms, such as spray dried, powdered, beaded forms, encapsulated forms, and mixtures thereof.

Suitable bulking agents include, but are not limited to maltodextrin (10 DE, 18 DE, or 5 DE), corn syrup solids (20 or 36 DE), sucrose, fructose, glucose, invert sugar, sorbitol, xylose, ribulose, mannose, xylitol, mannitol, galactitol, erythritol, maltitol, lactitol, isomalt, maltose, tagatose, lactose, inulin, glycerol, propylene glycol, polyols, polydextrose, fructooligosaccharides, cellulose and cellulose derivatives, and the like, and mixtures thereof. Additionally, granulated sugar (sucrose) or other caloric sweeteners such as crystalline fructose, other carbohydrates, or sugar alcohols can be used as a bulking agent due to their provision of good content uniformity without the addition of significant calories.

In one embodiment, the bulking agent may be a bulking agent described in U.S. Pat. No. 8,993,027.

In one embodiment, the bulking agent may be a bulking agent described in U.S. U.S. Pat. No. 6,607,771.

In one embodiment, the bulking agent may be a bulking agent described in U.S. U.S. Pat. No. 6,932,982.

In some embodiments, the tabletop sweetener composition may further comprise at least one anti-caking agent. As used herein the phrase “anti-caking agent” and “flow agent” refer to any composition which prevents, reduces, inhibits, or suppresses the at least one sweetener from attaching, binding, or contacting to another sweetener molecule. Alternatively, anti-caking agent may refer to any composition which assists in content uniformity and uniform dissolution. Non-limiting examples of anti-caking agents include cream of tartar, calcium silicate, silicon dioxide, microcrystalline cellulose (Avicel, FMC BioPolymer, Philadelphia, Pa.), and tricalcium phosphate. In one embodiment, the anti-caking agents are present in the tabletop sweetener composition in an amount from about 0.001 to about 3% by weight of the tabletop sweetener composition.

### Non-Animal Protein Materials and Products Made Therefrom

Products intended to replace or substitute meat or dairy products often rely on various non-animal-based materials, such as starches and proteins derived from plants, algae, fungi, or combinations thereof, to simulate the texture and flavor of meat or dairy. Non-limiting examples of non-animal-based proteins are plant proteins, such as soy proteins, pea proteins, bean proteins, grain proteins, and the like. Due to compositional differences between such plant-based materials and animal-derived materials, these proteins can impart a bitter taste.

Thus, in certain aspects, the disclosure provides a flavored product comprising a plant-based material (such as a plant-based starch, a plant-based protein, or a combination thereof) and the TM1 compound, or a comestibly acceptable salt thereof. In some embodiments, the flavored product comprises the TM1 compound, or a comestibly acceptable salt thereof, and a plant protein, such as pea protein, soy protein, potato protein, or any combination thereof. In some further embodiments, the flavored product can include any features of combination of features set forth above for ingestible compositions that contain the TM1 compound, or a comestibly acceptable salt thereof. In some embodiments, the flavored product is a beverage, such as soy milk, almond milk, rice milk, oat milk, a protein drink, a meal-replacement drink, or other like product. In some other embodiments, the flavored product is a meat-replacement product, such as a plant-based chicken product (such as a plant-based chicken nugget), a plant-based beef product (such as a plant-based burger), and the like. In some other embodiments, the flavored product is a protein powder, a meal-replacement powder, a plant-based creamer for coffee or tea, and the like. In certain further embodiments, any such products contain additional ingredients, and have additional features, as are typically used in the preparation and/or manufacture of such products. For example, such the TM1 compound, or comestibly acceptable salts thereof, may be combined with other flavors and taste modifiers, and may even be encapsulated in certain materials, according to known technologies in the relevant art. Suitable concentrations of the TM1 compound, or comestibly acceptable salts thereof, are set forth above.

In some embodiments, the flavored products comprise one or more plant-based proteins, which impart a bitter taste that is at least partially reduced by the use of the TM1 compound in the product. Such plant-based proteins include, but are not limited to, pea protein, soy protein, canola (rapeseed) protein, potato protein, chickpea protein, mycoproteins, algal proteins, fava protein, sunflower protein, wheat protein, and the like.

In some alternative embodiments analogous to the above embodiments, algal or fungal proteins or starches are used instead, oat protein, potato protein, and the like. In some embodiments, these flavored products also include fiber to provide texture to the product. Fibers suitable for use include, but are not limited to, psyllium fiber, pea fiber, potato fiber, curdlan, soluble corn fiber (dextran and/or maltodextrin), citrus fiber, and combinations thereof. In such products, the TM1 compound can be introduced in any suitable way. In some embodiments, the TM1 compound is incorporated into a flavoring emulsion, such as a water-in-oil emulsion, along with other flavor-imparting ingredients.

### Blocking Bitterness in Pharmaceutical APIs and Nutraceuticals

Many drug compounds impart a bitter taste, which therefore limits the ways in which they can be formulated and administered. Therefore, in certain aspects, the disclosure provides a nutraceutical or pharmaceutical composition comprising a bitter-tasting nutraceutical or pharmaceutical active ingredient, and the TM1 compound, or a comestibly acceptable salt thereof. Such nutraceutical or pharmaceutical compositions can be in any suitable form for oral administration, such as tablets, lozenges, capsules, powders, liquid solutions, liquid suspensions, and the like. Such nutraceutical or pharmaceutical compositions can include any suitable excipients, binders, and the like, such as those set forth in Remington's Pharmaceutical Sciences. In some embodiments, the bitter-tasting pharmaceutical active ingredient is an ion channel inhibitor, such as a proton channel inhibitor. Other examples of bitter-tasting APIs whose bitterness is reduced by TM1, or its pharmaceutically acceptable salts, include, but are not limited to, acetaminophen, atropine, brinzolamide, chloramphenicol, chloroquine, clindamycin, dexamethasone, digoxin, diltiazem, diphenhydramine, docusate, dorzolamide, doxepin, doxylamine, enalapril, erythromycin, esomeprazole, famotidine, gabapentin, ginkgolide A, guaifenesin, L-histidine, lomefloxacin, methylprednisolone, ofloxacin, oleuropein, oxyphenonium, pirenzepine, prednisone, ranitidine, trapidil, trimethoprim, and cetirizine.

### Use in Oral Care Products

Oral care products often contain ingredients that impart astringent or bitter off tastes. Such ingredients include menthol, menthol analogues, mint extracts, sodium bicarbonate, alkali metal salts of peroxymonosulfate (potassium peroxymonosulfate), cetylpyridinium chloride, lauramidopropyl betaine, cocamidopropyl betaine, arginine, hydrogen peroxide, chlorhexidine gluconate, zinc phosphate, zinc chloride, zinc citrate, potassium nitrate, pentasodium triphosphate, tetrasodium pyrophosphate, stannous fluoride, thymol, methyl salicylate, eucalyptol, or any combination thereof. Suitable oral care products include toothpaste, mouthwashes, whitening agents, dentifrices, and the like.

### EXAMPLES

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Example 1—Reduction in Bitterness of Acetaminophen

A series of test samples and a comparative sample were prepared. The comparative sample (C) contained acetaminophen in water at a concentration of 90 mM. Four test samples were prepared. Each test sample contained acetaminophen in water at a concentration of 90 mM. In addition, one test sample (S1) also contained TM1 at 100 ppm, another sample (S2) contained TM1 at 50 ppm, another sample (S3) contained TM1 at 25 ppm, and another sample (S4) contained TM1 at 10 ppm. Sample pairs, a test sample and a comparative sample, were presented in a blinded fashion to panelists, they were asked to taste the samples and identify the sample with the highest bitterness. Table 1 shows the results.

TABLE 1

| | Test Sample Chosen | C Chosen | Total Panelists |
|---|---|---|---|
| S1 | 4 | 27 | 31 |
| S2 | 9 | 39 | 48 |
| S3 | 12 | 33 | 45 |
| S4 | 14 | 31 | 45 |

### Example 2—Reduction in Bitterness of Guaifenesin

A test sample and a comparative sample were prepared. The comparative sample (C) contained guaifenesin in water at a concentration of 24 mM. The test sample (S1) contained guaifenesin in water at a concentration of 24 mM and TM1 at a concentration of 50 ppm. Sample pairs, a test sample and a comparative sample, were presented in a blinded fashion to panelists, they were asked to taste the samples and identify the sample with the highest bitterness. Table 2 shows the results.

TABLE 2

| | Test Sample Chosen | C Chosen | Total Panelists |
|---|---|---|---|
| S1 | 7 | 32 | 39 |

### Example 3—Reduction in Bitterness of Green Tea

A series of test samples and a comparative sample were prepared. The comparative sample (C) contained green tea extract in water at a concentration of 0.30 wt %. Two test samples were prepared. Each test sample contained green tea extract in water at a concentration of 0.30 wt %. In addition, one test sample (S1) also contained TM1 at 20 ppm, and another sample (S2) contained TM1 at 40 ppm. Sample pairs, a test sample and a comparative sample, were presented in a blinded fashion to panelists, they were asked to taste the samples and identify the sample with the highest bitterness. Table 3 shows the results.

TABLE 3

| | Test Sample Chosen | C Chosen | Total Panelists |
|---|---|---|---|
| S1 | 11 | 28 | 39 |
| S2 | 13 | 26 | 39 |

### Example 4—Reduction in Bitterness of Ginseng

A test sample and a comparative sample were prepared. The comparative sample (C) contained ginseng in water at a concentration of 0.036 wt %. The test sample (S1) contained ginseng in water at a concentration of 0.036 wt % and TM1 at a concentration of 10 ppm. Sample pairs, a test sample and a comparative sample, were presented in a blinded fashion to panelists, they were asked to taste the samples and identify the sample with the highest bitterness. Table 4 shows the results.

TABLE 4

| | Test Sample Chosen | C Chosen | Total Panelists |
|---|---|---|---|
| S1 | 13 | 26 | 39 |

### Example 5—Reduction in Bitterness of Caffeine

A test sample and a comparative sample were prepared. The comparative sample (C) contained caffeine in water at a concentration of 0.08 wt %. The test sample (S1) contained caffeine in water at a concentration of 0.08 wt % and TM1 at a concentration of 10 ppm. Sample pairs, a test sample and a comparative sample, were presented in a blinded fashion to panelists, they were asked to taste the samples and identify the sample with the highest bitterness. Table 5 shows the results.

TABLE 5

| | Test Sample Chosen | C Chosen | Total Panelists |
|---|---|---|---|
| S1 | 10 | 29 | 39 |

### Example 6—Reduction in Bitterness of Potato Protein

A test sample and a comparative sample were prepared. The comparative sample (C) contained potato protein in water at a concentration of 4 wt %. The test sample (S1) contained potato protein in water at a concentration of 4 wt % and TM1 at a concentration of 10 ppm. Sample pairs, a test sample and a comparative sample, were presented in a blinded fashion to panelists, they were asked to taste the samples and identify the sample with the highest bitterness. Table 6 shows the results.

TABLE 6

| | Test Sample Chosen | C Chosen | Total Panelists |
|---|---|---|---|
| S1 | 13 | 26 | 39 |

### Example 7—Reduction in Bitterness of Pea Protein

A test sample and a comparative sample were prepared. The comparative sample (C) contained pea protein in water at a concentration of 4 wt %. The test sample (S1) contained pea protein in water at a concentration of 4 wt % and TM1 at a concentration of 10 ppm. Sample pairs, a test sample and a comparative sample, were presented in a blinded fashion to panelists, they were asked to taste the samples and identify the sample with the highest bitterness. Table 7 shows the results.

TABLE 7

| | Test Sample Chosen | C Chosen | Total Panelists |
|---|---|---|---|
| S1 | 10 | 20 | 30 |

### Example 8—Reduction in Bitterness of Saccharin

A test sample and a comparative sample were prepared. The comparative sample (C) contained saccharin in water at a concentration of 1000 ppm. The test sample (S1) contained saccharin in water at a concentration of 1000 ppm and TM1 at a concentration of 10 ppm. Sample pairs, a test sample and a comparative sample, were presented in a blinded fashion to panelists, they were asked to taste the samples and identify the sample with the highest bitterness. Table 8 shows the results.

TABLE 8

| | Test Sample Chosen | C Chosen | Total Panelists |
|---|---|---|---|
| S1 | 9 | 24 | 33 |

### Example 9—Reduction in Bitterness of Grapefruit Juice

A test sample and a comparative sample were prepared. The comparative sample (C) consisted of grapefruit juice. The test sample (S1) consisted of grapefruit juice and TM1 at a concentration of 10 ppm. Sample pairs, a test sample and a comparative sample, were presented in a blinded fashion to panelists, they were asked to taste the samples and identify the sample with the highest bitterness. Table 9 shows the results.

TABLE 9

| | Test Sample Chosen | C Chosen | Total Panelists |
|---|---|---|---|
| S1 | 13 | 26 | 39 |

### Example 10—Reduction in Bitterness of Coffee

A test sample and a comparative sample were prepared. The comparative sample (C) instant coffee (Taster's Choice French Roast, acquired in the United States) made according to the directions. The test sample (S1) consisted of instant coffee (Taster's Choice French Roast, acquired in the United States) made according to the directions and TM1 at a concentration of 10 ppm. Sample pairs, a test sample and a comparative sample, were presented in a blinded fashion to panelists, they were asked to taste the samples and identify the sample with the highest bitterness. Table 10 shows the results.

TABLE 10

| | Test Sample Chosen | C Chosen | Total Panelists |
|---|---|---|---|
| S1 | 6 | 27 | 33 |

1. Use of a flavor-modifying composition to reduce a bitter taste of an ingestible composition, wherein the flavor-modifying composition comprises:
a first flavor-modifying compound, which is a compound of formula (I) 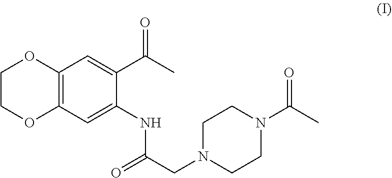 [SMILES: CC(=O)c1cc2c(cc1NC(=O)CN1CCN(C(C)=O)CC1)OCCO2]
or a comestibly acceptable salt thereof; and
wherein the ingestible composition comprises a bitter tastant.

2. The use of claim 1, wherein the bitter tastant is a non-animal protein.

3. The use of claim 1, wherein the non-animal protein is pea protein, soy protein, potato protein, chickpea protein, a bean protein, sunflower protein, or any combination thereof.

4. The use of claim 1, wherein the bitter tastant is an active pharmaceutical ingredient.

5. The use of claim 1, wherein the bitter tastant is a high-intensity sweetener.

6. The use of claim 1, wherein the bitter tastant is ginseng.

7. The use of claim 1, wherein the ingestible composition is coffee or tea.

8. The use of any one of claims 1 to 7, wherein the ingestible composition comprises an umami tastant.

9. The use of any one of claims 1 to 8, wherein the ingestible composition is a food product, a beverage product, an oral care product, nutraceutical, or a pharmaceutical product.

10. A method of reducing a bitter taste of an ingestible composition, the method comprising introducing a flavor-modifying composition to the ingestible composition, wherein the flavor-modifying composition comprises a first flavor-modifying compound, which is a compound of formula (I) 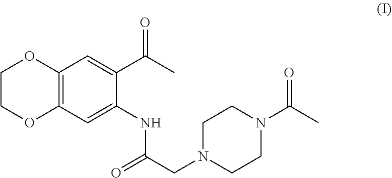 [SMILES: CC(=O)c1cc2c(cc1NC(=O)CN1CCN(C(C)=O)CC1)OCCO2]
or a comestibly acceptable salt thereof; and
wherein the ingestible composition comprises a bitter tastant.

11. An ingestible composition comprising a bitter tastant and a flavor-modifying compound, wherein the flavor-modifying compound is a compound of formula (I) 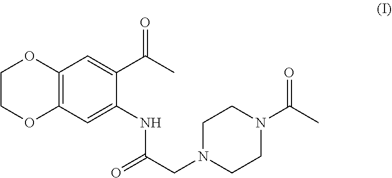 [SMILES: CC(=O)c1cc2c(cc1NC(=O)CN1CCN(C(C)=O)CC1)OCCO2]
or a comestibly acceptable salt thereof;
wherein the concentration of the flavor-modifying compound in the ingestible composition ranges from 1 ppm to 200 ppm, based on the total weight of the ingestible composition.

12. A flavored product, which comprises an ingestible composition of claim 10.

13. The flavored product of claim 11, which is a food product, a beverage product, an oral care product, nutraceutical, or a pharmaceutical product.
